(19) United States
(12) Patent Application Publication
(10) Pub. No.: US 2025/0229073 A1
(43) Pub. Date: Jul. 17, 2025

(54) TRANSIENT DISRUPTION OF THE NEUROVASCULAR BARRIER OF A HUMAN AND USES THEREOF FOR TREATING AMYOTROPHIC LATERAL SCLEROSIS

(71) Applicants: SORBONNE UNIVERSITE, Paris (FR); INSTITUT NATIONAL DE LA SANTÉ ET DE LA RECHERCHE MÉDICALE, Paris (FR); ASSISTANCE PUBLIQUE - HÔPITAUX DE PARIS, Paris (FR); CENTRE NATIONAL DE LA RECHERCHE SCIENTIFIQUE, Paris (FR); INSTITUT DU CERVEAU ET DE LA MOELLE EPINIERE, Paris (FR)

(72) Inventors: Alexandre CARPENTIER, Paris (FR); Anne-Sophie MONTERO, Paris (FR); Séverine BOILLEE, Paris (FR)

(73) Assignees: SORBONNE UNIVERSITE, Paris (FR); INSTITUT NATIONAL DE LA SANTÉ ET DE LA RECHERCHE MÉDICALE, Paris (FR); ASSISTANCE PUBLIQUE - HÔPITAUX DE PARIS, Paris (FR); CENTRE NATIONAL DE LA RECHERCHE SCIENTIFIQUE, Paris (FR); INSTITUT DU CERVEAU ET DE LA MOELLE EPINIERE, Paris (FR)

(21) Appl. No.: 18/850,522

(22) PCT Filed: Mar. 24, 2022
(86) PCT No.: PCT/EP2022/057844

§ 371 Date: Sep. 24, 2024

(51) Int. Cl. A61N 7/00

(52) U.S. Cl. CPC A61N 7/00; A61N 2007/0021; A61N 2007/0039; A61N 2007/0047

(57) ABSTRACT

The present disclosure provides a means for transiently disrupting the neurovascular barrier of a human. More particularly, the disclosure relates to an ultrasound contrast agent for use in treating Amyotrophic Lateral Sclerosis (ALS) in a subject, wherein the ultrasound contrast agent is used in combination with ultrasound beam for transiently disrupting the neurovascular barrier of the subject.

### TECHNICAL FIELD

The present invention relates to means for transiently disrupting the neurovascular barrier of a subject. More particularly, the invention relates to ultrasound contrast agent for use in treating Amyotrophic Lateral Sclerosis (ALS) in a subject, wherein the ultrasound contrast agent is used in combination with ultrasound beam for transiently disrupting the neurovascular barrier of the subject.

### BACKGROUND

Amyotrophic lateral sclerosis is a fatal neurodegenerative disease of the human motoneuron system which usually takes a lethal course within 3 to 5 years, whose causes have not been determined etiologically and for which there is no, or no significant, therapy as yet. The progressive decay of nerve cells of the first and second motor neurons are the cause of an increasing paralysis of the voluntary muscles, eventually leading to a total walking inability and the increasing paralysis of the respiratory musculature.

Worldwide, the prevalence of this disease is 4 in 100,000 and its incidence is 1 in 100,000 inhabitants. Median survival after onset of symptoms is between 24 and 48 months.

Very few products are being studied for ALS, particular examples being peptide compounds such as IGF-1 (Insulin-like Growth Factor 1) and BDNF (Brain Derived Neurotrophic Factor), which are described in Annals of Neurology, 1995, 38, 971, and Nature, 1992, 360, 753-759. The only non-peptide compound to have been tested for this disease is riluzole, whose chemical name is 2-amino-6-trifluoromethoxybenzothiazole, which is apparently capable of slowing down the progression of the disease in a particular group of subjects suffering from ALS (G. Bensimon et al., N. Engl. J. Med., 1994, 330, 585-591; Scrip, 1995, No. 2035:21), but no product effective in the treatment of this disease is currently available on the pharmaceutical market. According to the article by G. Bensimon et al. cited above, riluzole prolongs the survival of patients suffering from ALS, but the side effects, such as asthenia, spasticity and an increase in the transaminase levels, impair the quality of life of said patients.

It is well known that the blood brain barrier (BBB) and the blood spinal cord barrier (BSCB) are obstacles to the delivery of drugs to the central nervous system and may account for the lack of effective treatments for ALS. These barriers are formed by tight junctions of endothelial cells, astrocyte end-feet processes, with efflux transporters further limiting the penetration the majority of drugs. Although these barriers are partially dysregulated in ALS (endothelial cell degeneration, alteration of tight junctions), overexpression of active efflux transporters leads to limited additional accumulation of drugs, with riluzole brain distribution decreased by 1.7 in ALS models compared to wild type models.

Low intensity pulsed ultrasound can be used to transiently disrupt the BBB and BSCB to enhance drug penetration. When intravenous microbubbles are stimulated by ultrasound, they oscillate and induce a mechanical strain on the vessel wall, leading to disruption of tight junctions and down regulation of efflux transporters. This technique has been shown to be safe in recent clinical trials in patients with glioblastoma, Alzheimer's disease, and ALS. In the ALS clinical trial (Abrahao, A. et al. First-in-human trial of blood-brain barrier opening in amyotrophic lateral sclerosis using MR-guided focused ultrasound. Nat. Commun. 10, 4373 (2019)), the BBB was successfully disrupted in four ALS patients. However, up to now, no effective treatment for ALS has been developed.

Therefore, there is still a need for a non-invasive, safe and effective method of treating ALS.

### SUMMARY

The present invention provides a novel, safe and effective ultrasound way to treat ALS by transiently disrupting a neurovascular barrier of the subject. More particularly, the inventors have shown for the first time that transient and repetitive disruption of BBB and/or BSCB of a subject with ALS leads to great benefit for the subject. According to the present invention, transiently disrupting the BBB and/or BSCB, may allow a drug present in the systemic circulation to cross the BBB and/or BSCB and to target the brain. However, the inventors have shown that the repetitive disruption of the BBB and/or BSCB per se is beneficial in treating ALS. That is to say that treatment of ALS may occur by repetitively disrupting BBB and/or BSCB of the subject, even in absence of any drug administration.

The present invention therefore relates to an ultrasound contrast agent for use in treating Amyotrophic Lateral Sclerosis (ALS) of a subject in need thereof, by transiently disrupting a neurovascular barrier of the subject, wherein the ultrasound contrast agent is administered before and/or during the application to the central nervous system of the subject, of at least one ultrasound (US) beam, wherein at least five sessions of application of the at least one US beam are performed repetitively.

Advantageously, ultrasound contrast agent is administered before and/or during each application of US beam(s) to the central nervous system of the subject.

In a particular embodiment, the ultrasound (US) beam is applied to the spinal cord of the subject, in order to transiently disrupting the blood-spinal cord barrier (BSCB) of the subject.

In another embodiment, the US beam is applied to the brain of the subject, in order to transiently disrupting the blood brain barrier (BBB) of the subject.

In another embodiment, the US beam is applied both to the spinal cord and to the brain of the subject, in order to transiently disrupting both the BSCB and the BBB of the subject.

According to the invention, a therapeutically active agent may be administered before, during or after application(s) of the US beam(s). Alternatively, no therapeutically active agent is administered before, during or after application(s) of the US beam(s). In a particular embodiment, the ultrasound contrast agent is administered in absence of any therapeutically active agent.

Further features of the present invention will be apparent from the accompanying drawings and the following detailed description.

### BRIEF DESCRIPTION OF THE DRAWINGS

FIG. 1. (A) Disease progression in SOD1^{G93A} ALS mice and treatment protocol. Typical evolution of the weight of a mutant SOD1^{G93A} female mouse modeling ALS. The peak weight defines the onset of symptomatic disease. The five sessions of treatment (stars) with ultrasound, IGF1, or the combination of the two were performed weekly from Days 107 to Day 143 for each mouse. (B) Schematic of treatment setup. Ultrasound transducer was coupled to the skin with ultrasound coupling gel and positioned to target the lumbar spinal cord.

FIG. 2. Evan's blue and IGF1 infiltrate the spinal cord after sonication. (A) Macroscopic view of a sonicated spinal cord of a wild-type mouse after Evan's blue dye (EBD) injection. The blue mark at the sonicated segment of the spinal cord corresponds to a local passage of the EBD in the central nervous system after blood-spinal cord barrier disruption. (B) Concentration of Evan's blue dye in the spinal cord of control and sonicated (US) wild type mice (left). Concentration of IGF1 in the spinal cord of control and sonicated (US) mice (right). IFG1 concentration was 1100 times higher in the sonicated segments of spinal cord. ***=p=0.001, ****=p<0.0001

FIG. 3. Sonication increases survival of ALS mice. (A-B) Kaplan-Meier curves for (A) the age of disease onset of the mice in the different treatment groups or (B) survival, for the different groups of treated mice. Control: SOD1^{G93A} mice, US: sonicated SOD1^{G93A} mice, IGFI: IGFI-treated SOD1^{G93A} mice, US IGFI: IGFI-treated and sonicated SOD1^{G93A} mice. n represent the number of mice in each group and is given with the median+/−SEM age in days. (C-D) Impact of treatment on (C) early phase disease duration and (D) late phase disease duration. Bars: median±SEM. *p<0.05, **p<0.01, ***p<0.001, ns=statistically non-significant

FIG. 4. Sonication modifies glial cell reactivity and lymphocyte infiltration. Histological analysis of spinal cord of the ALS treated mice at the end of the early disease phase (symptomatic stage defined by 10% of weight loss) and disease end stage. (A) quantification of mutant SOD1 by immunostained area. (B) Motor neuron counts. (C) Astrocyte reactivity measured by % GFAP immunostained area. (D) Microglial reactivity measured by % Iba1+ immuno-positive area. (E) Lymphocyte infiltration measured by counting the number of CD4+ and CD8+ cells per spinal cord slice. Bars: Mean+/−SEM *p<0.05, **p<0.01, ns=statistically non-significant. N=number of mice

### DETAILED DESCRIPTION

The invention relates to ultrasound contrast agent for use for transiently disrupting a neurovascular barrier of the subject, particularly of a human, wherein the delivery of ultrasound contrast agent is combined with the application of ultrasound beam(s) to the central nervous system of the subject and wherein the application of US beams is performed repetitively at least five times. The combined administration of ultrasound contrast agent and US beams, repetitively to the central nervous system of the subject with ALS leads to a repeated transient disruption of the neurovascular barrier of the subject and provides great benefit. Particularly, an increase in survival and slowing down of motor neuron loss is observed. This repeated transient disruption of the neurovascular barrier may be further combined to the administration of therapeutically active agent to further treat ALS.

The present disclosure will best understood by reference to the following definitions.

### Definitions

“The central nervous system” refers to the part of the nervous system consisting of two major structures, namely the brain and the spinal cord.

In the context of the invention, the term “neurovascular barrier” of a subject refers to semipermeable interface of endothelial cells that prevents solutes in the circulating blood from non-selectively crossing into the extracellular fluid of the central nervous system. Particularly, the neurovascular barrier includes the blood brain barrier (BBB) and the blood-spinal cord barrier (BSCB).

In the context of the invention, the term “disrupting the neurovascular barrier, BBB or BSCB”, “opening the neurovascular barrier, BBB or BSCB” or “increasing the permeability of the neurovascular barrier, BBB or BSCB” are used to refer to an increased susceptibility of the neurovascular barrier, BBB or BSCB to the passage of molecules and agents (such as Lymphocytes) there through that occurs without detectable damages of the central nervous system.

In the context of the invention, a “transient” opening refers to a reversible opening occurring preferably for more than 1 hour, the neurovascular barrier returning after that to its initial state, i.e., state before the application of the first US beam. Generally, the opening occurs for a period of time from 1 to 48 hours, preferably from 5 to 24 hours, more preferably from 6 to 10 hours, such as for approximately 8 hours.

The term “ultrasound contrast agent” is used herein to refer to a substance (solid, liquid or gas) that is able to enhance the contrast between the region containing the agent and the surrounding tissue in an ultrasound image. Advantageously, the ultrasound contrast agent corresponds to small bubbles of a gas, termed “microbubbles,” with an average diameter between 1 μm and 20 μm. Said microbubbles oscillate and vibrate when US is applied and may reflect ultrasound waves. The ultrasound contrast agent is generally injected intravenously into the blood stream, wherein it remains for a limited period of time.

The term “ultrasound beam”, “ultrasound wave” and “ultrasound” are used indifferently for designating sound waves with frequencies higher than 200 kHz.

As used herein, “subject” refers to a “human”, i.e., a person of the species Homo sapiens, including man, woman, child and human at the prenatal stage. In one embodiment, a subject may be a “patient” who is awaiting the receipt of, or is receiving medical care or was/is/will be the object of a medical procedure, or is monitored for the diagnosis or the development of a disease.

In the context of the invention, the terms “treatment”, “treat” or “treating” are used herein to characterize a therapeutic method or process that is aimed at (1) slowing down or stopping the progression, aggravation, or deterioration of the symptoms of the disease state or condition to which such term applies; (2) alleviating or bringing about ameliorations of the symptoms of the disease state or condition to which such term applies; and/or (3) reversing or curing the disease state or condition to which such term applies.

A “therapeutically effective amount” or “efficient concentration” refers to mean levels or amount of substance that is aimed at, without causing significant negative or adverse side effects to the target, delaying or preventing the onset of a disease, disorder, or condition related to ALS; slowing down or stopping the progression, aggravation, or deterioration of one or more symptoms of ALS; bringing about ameliorations of the symptoms of the disease, disorder, or condition related to ALS; reducing the severity or incidence of ALS; or curing ALS. A therapeutically effective amount may be administered prior to the onset of ALS, for a prophylactic or preventive action. Alternatively or additionally, the therapeutically effective amount may be administered after onset of the disease, for a therapeutic action.

Throughout the disclosure, various aspects of the invention can be presented in a range format. It should be understood that the description in range format is for convenience and brevity and should not be constructed as an inflexible limitation on the scope of the invention. Accordingly, the description of a range should be considered to have specifically disclosed all the possible subranges as well as individual numerical values within that range, range values being included.

### Application of Ultrasound Beams

The purpose of the present invention is to provide means for treating ALS in a subject in need thereof. To this end, the present invention propose to repetitively disrupt, in a transient way, the neurovascular barrier of the subject. Indeed, the inventors have shown that by transiently disrupting the neurovascular barrier of a subject with ALS, conditions of the subject can be improved. Particularly, the inventors have shown that beneficial effects on ALS may be obtained after five sessions of US beam(s) applications to the central nervous system of the subject.

According to the invention, the neurovascular barrier may be disrupted by both applying ultrasound (US) beam(s) to the central nervous system a human subject, and administering intravenously an ultrasound contrast agent. More particularly, the US beam is applied repetitively, at least five time, to central nervous system in order to disrupt repetitively the neurovascular barrier of the patient. Advantageously, at least 5 sessions of application of US beam are performed repetitively. Particularly, between 5 and 50 sessions, or more, of application of US beam are performed, such as between 5 and 40, between 5 and 30, between 5 and 25, between 5 and 20, between 5 and 15, between 5 and 10. Particularly, 5, 7, 10, 12, 15, 17, 20, 22, 25, 30, 35, 40, 45 or 50 sessions of application of US beam are performed repetitively.

The lag period between two successive sessions may be of at least 24 hours, preferably of several days. Advantageously, the lag period between two successive sessions is of 1 week or more. Particularly, the lag period may be between at least 1 week and at most 9 weeks, such as 2 weeks, 3 weeks, 4 weeks, 5 weeks, 6 weeks, 7 weeks, 8 weeks or 9 weeks. For instance, the lag period may be of 7 days, 10 days, 15 days, 22 days, 25 days, 30 days, 35 days, 40 days, 45 days. Preferably, the lag period between two successive sessions doesn't exceed 60 days.

The lag period between two successive sessions may be same for all the sessions of application of US beams. Alternatively, the lag period may vary during the treatment. For instance, the lag period between two successive sessions may be shorter at the begging of the treatment than at the end of the treatment, or the reverse. In a particular embodiment, the lag period between two successive sessions at the beginning of the treatment is between 7 and 30 days, such as between 15 and 20 days, and the lag period between two successive sessions at the end of the treatment is between 30 and 60 days.

In a particular embodiment, US beam are applied repetitively 20 times, with a lag period between two successive sessions of 15 days.

According to the invention, the treatment may be performed several time. That is to say that the subject receives a first set of sessions of application of US beams, in order to disrupt repetitively and transiently the neurovascular barrier during a first period of time. And, one or more additional set of sessions of application of US beams may be performed again later, if need be.

For instance, a first set of 5 to 30 sessions of application of US beams is applied during a first period of time, preferably about 20 sessions, corresponding to an attack treatment. During this attack treatment, the lag period between two successive period may be between 7 and 60 days, preferably about 2 weeks. Then a second set of 5 to 20 sessions of application of US beams is applied during a second period of time, corresponding to a maintenance treatment. This attack treatment spread over a time period of months or years. During this attack treatment, the lag period between two successive period may be between 1 month and 3 or more months. For instance, at the begging of the maintenance treatment, the lag period is about 1 month, for 6 months, and then the lag period is increased up to 3 months, for 1 to 4 years or more.

Advantageously, at least one session of application of US beam(s) comprises application of several US beams sequentially or in synchrony, for a period of time between 100 and 500 seconds, preferably for a period of time of 250 seconds. In a particular embodiment, most of the at least five sessions of application, preferably all the sessions application comprises application of several US beams sequentially or in synchrony, for a period of time between 100 and 500 seconds, preferably for a period of time of 250 seconds.

The US beams may be applied to the spinal cord of the subject, in order to transiently disrupting the BSCB of the subject. Particularly, the US beam may target a cervical portion of the spinal cord of the subject and/or a lumbar portion of the spinal cord of the subject. For instance, the US beam may target a portion between neurological metameric segments C3 and C7. Alternatively or in addition, the US beam may target a lumbar portion of the spinal cord of the subject, preferably a portion between neurological metameric segments L1 and S4.

Advantageously, the US beams are applied by use of a US transducer that has been previously implanted inside the spinal canal of the spine of the subject, preferentially in the subdural and/or in the epidural space of the spinal canal. Examples of suitable US transducer are disclosed in PCT/EP2020/058833, PCT/IB2016/000430. Of course, an external US transducer may be used either. For instance, an external US transducer may be positioned against the back of the subject, substantially parallel to the elongation line of the spine. Examples of suitable US transducer are disclosed in PCT/IB2016/000431 or Montero, A.-S. et al. 2019 (Ultrasound-Induced Blood-Spinal Cord Barrier Opening in Rabbits. Ultrasound Med. Biol. 45, 2417-2426).

Alternatively or in addition, the US beams may be applied to the brain of the subject, in order to transiently disrupting the BBB of the subject. Particularly, the US beams may target a region within the right and/or left frontal lobes, more particularly a region within the primary motor cortex area and/or the supplementary motor cortex area (Hynynen, K., McDannold, N., Vykhodtseva, N. & Jolesz, F. A. Noninvasive MR imaging-guided focal opening of the blood-brain barrier in rabbits. Radiology 220, 640-646 (2001), Abrahao, A. et al. First-in-human trial of blood-brain barrier opening in amyotrophic lateral sclerosis using MR-guided focused ultrasound. Nat. Commun. 10, 4373 (2019)).

Advantageously, the US beam are applied by use of a US transducer that has been previously implanted within a burr hole in the skull of the subject. Examples of suitable implantable US transducer for brain are disclosed in U.S. Ser. No. 13/577,938, WO201609786, WO2018234280, and Carpentier, A. et al. 2016 (Clinical trial of blood-brain barrier disruption by pulsed ultrasound. Sci. Transl. Med. 8, 343re2). Of course, an external US transducer may be used either. Examples of suitable external US transducer for brain are disclosed in Abrahao, A. et al. 2019 (First-in-human trial of blood-brain barrier opening in amyotrophic lateral sclerosis using MR-guided focused ultrasound. Nat. Commun. 10, 4373)

In some embodiments, the nervous barrier disruption is delimited, i.e., occurs solely in a target region of the BBB and/or BSCB. Preferably, at least 5 cm³, preferably at least 10 cm³, more preferably at least 20 cm³ of the BBB and/or BSCB is disrupted. In other embodiment, the BBB and/or BSCB disruption is generalized.

According to the invention, the US beams are focused or unfocused, preferably unfocused.

Advantageously, the unfocused US beams are applied to the spinal cord and/or brain of the subject, with a pressure level ranging from 0.3 to 2 MPa, preferably from 0.3 to 1.2 MPa. In the context of the invention, the “pressure level” refers to the maximum acoustic pressure measured in the acoustic field of the emitter in water. Advantageously, the unfocused US beams are applied within a pressure range of 0.7 MPa to 1.25 MPa, preferably within a pressure range of 0.8 MPa to 1.1 MPa, more preferably of about 1 MPa.

In the context of the invention, the value of the pressure level corresponds to the value of the pressure level onto the spinal cord and/or brain. In particular embodiment, the pressure coming out of the emitter may be higher, in order to take into account attenuation due to intervening tissues. Generally speaking, such attenuation may be at most of 30%.

According to the invention, the resonance frequency of the unfocused US beam preferably ranges from 0.5 to 3 MHz, such as from 1 to 1.1 MHz, particularly at 1.05 MHz. In a particular embodiment, the frequency of the unfocused US beam is approximately 1 MHz. In another particular embodiment, the frequency of the unfocused US beam is approximately 2 MHz.

In an embodiment, the unfocused US beam is applied in pulses of duration ranging from 10 to 300 ms and with a pulse repetition frequency ranging from 0.3 to 3 Hz, preferably from 0.5 to 1 Hz.

Unfocused US beams may be applied in pulses of duration ranging from 10 to 50 msec, preferably from 20 to 30 msec, more preferably about 25 msec, and with a pulse repetition frequency ranging from 0.3 to 1.2 Hz, preferably at 1 Hz.

Alternatively or in addition, the at least one US beam may be applied in pulses of duration ranging from a mechanical index of 0.1 to 2 and a duty cycle of less than 5%, and preferably a mechanical index 1.03 and a duty cycle of 1.2%. For periodic events, such as ultrasound pulses, duty cycle represents the ratio of the duration of a pulse on the total duration of a period. This ratio is here expressed as a percentage. In the context of the invention, the mechanical index refers to the peak negative pressure in situ (MPa) divided by the square root of the frequency (MHz).

Advantageously, the unfocused US beams are applied for a short period of time, such as less than 5 minutes, preferably approximately 4 minutes.

### Ultrasound Contrast Agent

The method of the invention further requires the presence of an ultrasound contrast agent in the area of the neurovascular barrier, e.g., in the area of the BBB and/or BSCB. The US contrast agent may be administered by injection, preferably by systemic injection. Systemic administration is a route of administration of an agent into the circulatory system so that the entire body is affected. Examples of systemic injections include intravenous, subcutaneous, intramuscular, intradermal, intravitreal, or perfusion.

In some embodiments, the ultrasound contrast agent is injected into the bloodstream of the subject.

Preferably, the ultrasound contrast agent is administered as a bolus just before the US beam application. More preferably, the US contrast agent is administered between 0 and 30 seconds before the US beam application. Advantageously, the US beam application and the US contrast agent administration are concomitant. When several US beams are applied in a same session, the ultrasound contrast agent is preferably delivered only once, concomitantly with the first US beam application, though it may be delivered by a continuous infusion through the activation of successive US beams. Preferably, ultrasound contrast agent is administered for each session of application of US beam(s).

According to the invention, the ultrasound contrast agent may contain gaseous bubbles, a high concentration of gas, solid particles configured to vaporize in response to ultrasound, liquid configured to vaporize in response to ultrasound, micro particles configured to act as cavitation sites, solid particles having higher acoustic impedance than tissue in the desired region, and/or liquid with a high acoustic absorption coefficient.

In some embodiments, the ultrasound contrast agent is a microbubble contrast agent, preferably selected from the group consisting of sulphur hexafluoride microbubbles (SonoVue®), microbubbles made of an albumin shell and octafluoropropane gas core (Optison®), perflexane microbubbles encapsulated in an outer lipid shell (Imagent®), microbubbles made of octafluoropropane gas core encapsulated in an outer lipid shell (Definity®), or perfluorobutaine and nitrogen gas encapsulated in a lipid shell (BR38-Schneider et al., 2011). Preferably, the ultrasound contrast agent consists of sulphur hexafluoride microbubbles.

The microbubbles may have a mean diameter in a range from 1 μm to 20 μm. In some embodiments, the microbubbles have a mean diameter in a range from 4 μm to 5 μm. In some other embodiments, the microbubbles have a mean diameter in a range from 2 to 6 μm. In some embodiments, the microbubbles have a mean diameter of approximately 7 μm, 6 μm, 5 μm, 4 μm, 3 μm or 2 μm. In a particular embodiment, the microbubbles have a mean diameter of approximately 2.5 μm.

In some embodiments, the dose of ultrasound contrast agent ranges between 0.01 and 0.4 ml/kg based on the total weight of the subject, preferably from 0.05 and 0.2 ml/kg. In a particular embodiment, the maximum dose of ultrasound contrast agent is up to 5 ml, up to 6 ml, up to 10 ml, up to 15 ml, up to 20 ml, up to 25 ml, or up to 30 ml. More generally, the maximum dose may depend of the dilution of the ultrasound contrast agent. Preferably, the dose of ultrasound contrast agent is approximately the dose used for diagnostic imaging, with a maximum dose corresponding to twice the dose used for diagnostic imaging.

In some embodiment, the ultrasound contrast agent is administered in absence of any therapeutically active agent, or drug. Indeed, the inventors have shown that beneficial effects may be obtain in the treatment of ALS by disrupting the neurovascular barrier as disclosed above, even in absence of any therapeutically active agent, or drug. In a particular embodiment, no therapeutically active agent or drug is administered to the subject before, during or after disruption of the neurovascular barrier.

In some embodiments, a therapeutically active agent is used together with the ultrasound contrast agent. The therapeutically active agent is a drug that must be delivered to the spinal cord and/or brain of the patient in order to treat ALS. The therapeutically active agent is administered either orally or by injection, preferably by systemic injection. In a particular embodiment, the therapeutically active agent and the ultrasound contrast agent are administered sequentially. The ultrasound contrast agent may be administered within a suitable time window after or prior to the administration of the therapeutically active agent. For example, the ultrasound contrast agent is administered at the peak blood concentration of the therapeutic agent.

In some embodiments, the ultrasound contrast agent is administered days after the administration of the therapeutically active agent. In one example, the ultrasound contrast agent is administered 7 days after immunoregulator therapeutic agent such as Interleukine, in order to leave time for therapeutic agent to impact systemic circulating lymphocytes (CD4 TReg increase). Then the ultrasound contrast agent injection concomitant with ultrasound emission will allow protecting lymphocytes to enter the neurovascular barrier.

Alternatively, the ultrasound contrast agent and the therapeutically active agent may be administered concomitantly, or simultaneously, (e.g., by way of a same solution).

Alternatively, the therapeutically active agent may be encapsulated in the ultrasound contrast agent.

The “therapeutically active agent”, as used herein include any drug medicament, antibodies, glycoproteins, dissolution compounds, genetic materials such as RNA and DNA, stem cells, proteins or peptides (for ex antisens oligonucleotides), liposomes, lipids, synthetic or natural polymers or polymeric conjugates, macromolecules, nanocarriers, encapsulated drug molecules, interleukines, modified lymphocytes, antibodies (for ex antiTDP43), pharmaceutical formulations, any other substance capable of producing therapeutic actions, and any mixtures thereof.

More particularly, in the context of ALS treatment, the therapeutically active agent preferably includes oligonucleotides, interleukines, modified lymphocytes, growth factors . . . .

### Neurovascular Barrier Disruption

The application of US beams to the central nervous system of a subject in the presence of an ultrasound contrast agent injected to the subject leads to the transient opening of the neurovascular barrier, particularly of the BBB and/or BSCB. In some embodiments, the BBB and/or BSCB opening occurs for a period of time from 1 to 24 hours, preferably from 5 to 12 hours, more preferably from 6 to 10 hours. In some embodiments, the BBB and/or BSCB opening occurs for approximately 8 hours.

The disruption may be confirmed and/or evaluated by magnetic resonance imaging (MRI), blood biomarkers, electrophysiology or any other mean. For example, a gadolinium-based magnetic resonance (MR) contrast agent such as Dotarem® (gadoterate meglumine, Guerbet USA), which does not normally cross the BBB or BSCB, can be used to visualize the region of the neurovascular barrier disruption. When the agent is injected in a patient, a T1w MR sequence can be used to visualize regions of hypersignal and therefore visualize the effect of neurovascular barrier disruption by US. BBB and/or BSCB disruption typically leads to a change of 5-10% or more in MR signal enhancement after contrast agent administration. In addition, dynamic contrast enhanced (DCE) MR imaging techniques can be used to calculate the permeability of the neurovascular barrier and to quantify the magnitude of the permeability enhancement after ultrasound treatment.

The BBB and/or BSCB is then transiently disrupted, and against all odds, this transient disruption per se leads to great benefit in patient with ALS, as it is shown for the first time in the following results. In addition, this transient disruption may allow to molecules, such as drugs, to cross BBB and/or BSCB and to target the tissues of the central nervous system.

Therefore, an exogenous therapeutically active agent may be administered to the subject in addition to the ultrasound contrast agent, in order to enhance the beneficial effect of transient disruption of the neurovascular barrier in a subject with ALS.

### Treatment of ALS

The present invention relates to ultrasound contrast agent for use in treating ALS by transiently disrupting the BBB and/or BSCB of a human, wherein the ultrasound contrast agent is administered before or during the application, to the central nervous system of the human, of an unfocused ultrasound (US) beam.

According to the invention, treatment of ALS may be obtained by disruption of the BBB and/or BSCB of the subject, in absence of any therapeutically active agent. Particularly, the ultrasound contrast agent is administered in absence of any therapeutically active agent. In a particular embodiment, no therapeutically active agent is administered to the subject before, during and/or after application of the US beam(s). In the context of the present invention, “before” or “after” application of US beam(s) means that no therapeutically active agent is administered to the subject during a period of at least 1 hour, preferentially at least 1 day, more preferentially at least 1 week before and/or after application of US beam(s).

The present invention also relates to therapeutically active agent for use in treating ALS in a human, wherein the therapeutically active agent is to be delivered in addition to an ultrasound contrast agent, which is administered after, before or during the application, to the central nervous system of the human, of an unfocused ultrasound (US) beam in order to transiently disrupting the BBB and/or BSCB of the human, to allow the therapeutically active agent to cross the BBB and/or BSCB and to target the central nervous system. For instance, the ultrasound contrast agent is administered in conjunction with a therapeutically active agent. In another embodiment, the therapeutically active agent is administered to the subject either before, after or during disruption of the neurovascular barrier (local action), or between two disruption sessions (systemic action).

A method of treating a subject suffering from ALS is also provided, which comprises: administering to the subject an ultrasound contrast agent within a suitable time window after or prior to the application of the US beam to the central nervous system. Such method may be combined with the administration, in sequence or concomitantly with the ultrasound contrast agent, of a therapeutically active agent suitable to treat or prevent ALS.

Particularly, the inventors have shown that repetition of at least 5 sessions of US beam application on the central nervous system of a mice ALS model leads to a significant improvement of neurological conditions and improvement of survival of more than 12 days. Comparing the evolution speed of the disease in mice, equivalent survival improvement in patient is expected to be three months for 5 sessions, and then at least 12 months for 20 sessions. A long-lasting beneficial effect is expected and could increase patients' survival.

Particularly, a protocol of treatment including 5 or more sessions of US beam application, wherein two successive sessions are spaced of at least 15 days, leads to significant benefits.

In a particular embodiment, the protocol of treatment comprises 10 sessions of US beam application, wherein two successive sessions are spaced of about 15 days (attack treatment), 5 sessions of US beam application, wherein two successive sessions are spaced of about 30 days, and 5 sessions of US beam application, wherein two successive sessions are spaced of about 3 or 4 months.

The invention is particularly useful to treat ALS at an early stage of the disease to protect alive motor neurons and then maintain neurological condition. A late-stage treatment is also envisioned but may be less efficient due to already significant motor neuronal loss.

### EXAMPLES

### Material & Methods

### Animal Models

Eighty Mutant SOD1^{G93A} female mice modeling ALS [B6.Cg-Tg (SOD1^{G93A}) 1Gur/J] were purchased from Jackson Laboratories (stock #004435) and 120 wild type control mice (WT), with a C57B16/J genetic background. The animal protocol complied with the European laws for the protection of experimental animals and was approved by the local ethics committee. Mice were maintained on a 12:12 h dark: light cycle with food and water available ad libitum. Moisturized food pellets were supplied when mice became weak. ALS mice were hemizygous for a 12 kb genomic fragment encoding the human wild-type or mutated SOD1 gene under its endogenous promoter. Mice carrying the human SOD1 transgene were identified by PCR screening of tail DNA using mouse SOD1 forward primer (5′-GTTACATATAGGGGTTTACTTCATAATCTG-3′), human SOD1 forward primer (5′-CCAAGATGCTTAACTCTTGTAATCAATGGC-3′) and mouse and human SOD1 reverse primer (5′-CAGCAGTCACATTGCCCAGGTCTCCAACATG-3′) resulting in a ˜800 bp mouse and a ˜600 bp human SOD1 PCR product.

To represent as close as possible the clinical disease progression in humans, treatment started at the beginning of the disease and not at a presymptomatic stage. SOD1^{G93A} mice are a particularly severe model in terms of symptoms. The mice quickly become too weak to tolerate general anesthesia, which led us to stop treatments at 135 days of age, or 1 month of treatment (5 sessions). This corresponds approximately to the end of the early phase of the disease.

### Ultrasound Procedure

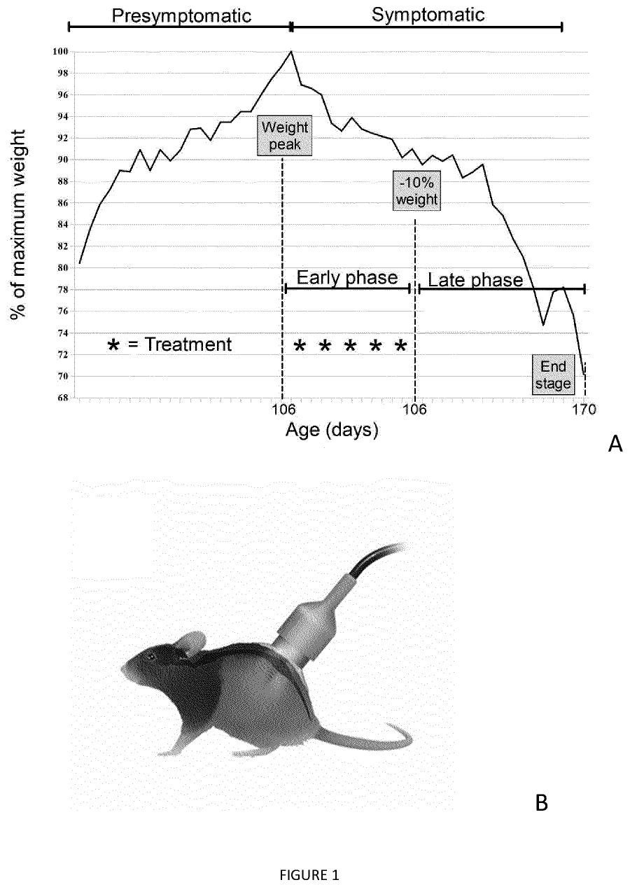

A 1-cm, 1 MHz planar ultrasound transducer was coupled to the skin with ultrasound coupling gel and positioned to target the lumbar spinal cord. The peak acoustic pressure was 0.5 MPa for tolerance tests in WT mice and 0.35 MPa was sufficient for blood spinal cord disruption assessment and for ALS mouse procedures (FIG. 1). At the beginning of the sonication, 0.2 mL of microbubbles (SonoVue, Bracco®) were injected intravenously. Twenty-four mice were used to test the sonication protocol

### Experimental Protocol in WT Mice

### Measurement of BSCB Opening by Evan's Blue Dye

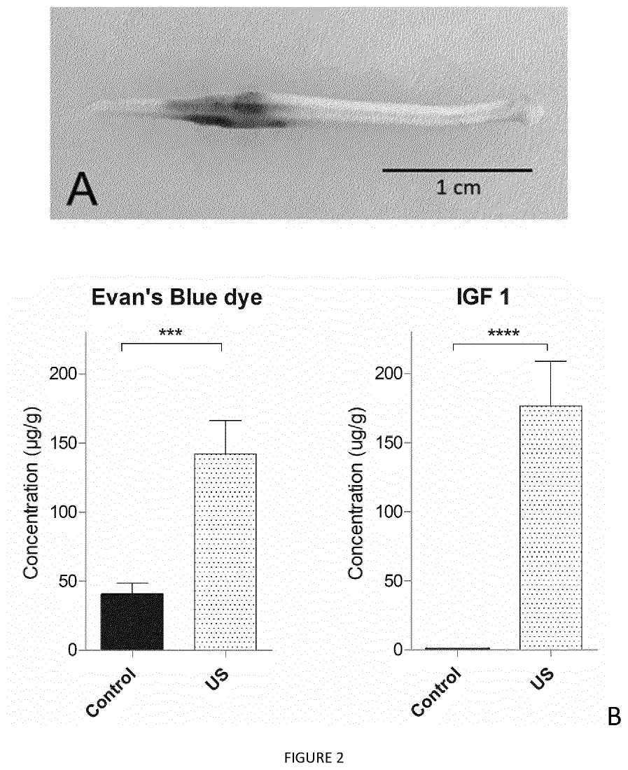

Blood spinal cord opening was assessed on 27 WT mice with Evan's Blue Dye (EBD) spinal cord uptake quantification. A representative example of BSCB opening with Evan's blue is shown in FIG. 2.

### Measurement of IGF1

IGF1 spinal cord uptake quantification was assessed on 29 WT mice. IGF1 measurements were performed using the Quantikine ELISA human IGF-1, R&D Systems kit, according to the manufacturer's instructions. Results are reported as μg of IGF1 per gram of protein. Fourteen sonicated lumbar spinal cord were compared to 15 non-sonicated lumbar spinal cord

Tolerance of BSCB opening: The tolerance of unique spinal cord sonication was assessed on 20 WT mice and repeated sonications on 20 WT mice: 10 treated with ultrasound once a week for five weeks compared to 10 non-sonicated control mice.

### Experimental Protocol in ALS Mice

ALS mice were randomly assigned to one of four groups: no treatment (Control, n=15), ultrasound (US, n=25), subcutaneous injections of IGF1 alone (IGF1 n=15), ultrasound and IGF1 (US IGF1, n=25). IGF1 and US IGF1 mice received a subcutaneous injection of 500 μg/kg rhIGF1 (Mecasermine—IPSEN Pharma®). Controls and US mice received a subcutaneous injection of 0.9% saline. US and US-IGF1 mice received sonication 30 minutes after IGF1 injection.

The onset of disease was defined as the time point at which the mice were at peak weight before they started to lose weight due to progressive motor neuron denervation and muscle wasting, as shown in FIG. 1. The symptomatic stage was defined as the age at which the mice had lost 10% of their maximal weight and is accompanied by gait alterations and failure of hindlimb splaying reflex. This disease stage was followed by progressive paralysis. A total of 45 mice were followed until death (n=10 control, n=10 US, n=10 IGF1 and n=15 US+IGF1 mice). In each group some randomly assigned mice were sacrificed at the symptomatic stage (defined as 10% of weight loss) or at disease end stage for immunohistological analysis.

Intervention (Control, US, IGF1, US IGF1) started for each mouse at P106 (corresponding to the mean age of disease onset), once a week, for 5 consecutive weeks. Mice were anesthetized with an intraperitoneal mixture of ketamine (100 mg/kg) and xylazine (10 mg/kg). Body temperature was maintained using a heating pad. Back hair was shaved to minimize air trapping between the skin and ultrasound emitter. Controls and IGF1 only mice received intravenous injection of saline instead of microbubbles and no ultrasound

### Clinical and Survival Evaluation

Muscle strength of mice was assessed by three different motor tests: grip testing, inverted screen testing and wire suspension testing. Variation of weight is classically used as an unbiased measure of disease course in this ALS mouse model. We defined peak weight as start of disease onset. The phase of early disease progression corresponded to the phase from peak weight to 10% loss of maximal weight. The late phase corresponded to the period between the 10% weight loss and the end stage of the animal. Starting at eight weeks, mice were weighed three times a week and daily when they approached the limit points (−10% weight, end stage). End stage, when mice were sacrificed, was defined by the mouse's incapacity to straighten itself within 30 seconds when placed on its side, an endpoint frequently used for mutant SOD1-expressing ALS mice. Neurological examination was performed before and after each procedure

### Sacrifice, Tissue Preparation and Immunohistology

Mice were anesthetized with an intraperitoneal injection of pentobarbital (400 mg/kg).
For EBD and IGF1 quantification (fresh tissues), the mice were perfused transcardially with 20 mL of PBS to wash the residual intravascular EBD or IGF1. The spinal cord was removed and frozen in liquid nitrogen.
For immunohistological analysis and motor neuron count (fixed tissues), the mice were perfused transcardially with 20 mL PBS then 50 mL of 4% paraformaldehyde in phosphate buffer. The spinal cord was extracted, and placed for 4 hours in 4% paraformaldehyde for post-fixation and then cryopreserved in a 30% sucrose solution in PBS before freezing. Thirty-micron thick transverse sections were cut on a cryostat at −18° C. Floating segments of spinal cord were stored in phosphate buffered saline solution at 4° C. Motor neuron counts were determined on serial sections across the lumbar spinal cords (15-20 sections per animal) after Nissl staining.
For immunostaining, sections were incubated overnight at room temperature with the following antibodies: rabbit anti-Iba1 (1:500 Wako Chemicals), anti-GFAP (1:500 Dako anti GFAP), rat anti-CD4 (1:100, AbD Serotec), rat anti CD3 (1:100, AbD Serotec), rat anti CD8 (1:100, AbD Serotec, rabbit polyclonal to human SOD1 (1:10000, Abcam). Species specific Alexa Fluor 488, 594, 647 (1:1000 Life Technologies) were used as secondary antibodies

### Statistical Analysis

GraphPad (GraphPad Software, San Diego, CA) was used for all statistical analysis. Statistical significance was set at p≤0.05. EBD and IGF1 concentrations were checked for normality and then compared using Student's t-test. Duration of early and late phases between the groups were compared by ANOVA after a normality test and followed by post-hoc Tukey-Kramer Multiple comparison test or Dunnett's multiple comparison test. Motor neuron count, immune activation and SOD1 expression were compared in different group using Kruskall Wallis test. Data are represented as Mean+SEM. Kruskal Wallis non parametric test was used to compare the total number of lymphocytes (CD4 and CD8). Survival analysis (onset disease, end-stage) was performed with Kaplan-Meier statistics. A Log-rank P value was calculated with Mantel-Cox test to compare the survival curves and corrected with the Bonferroni coefficient.

### Results

### Blood Spinal Cord Barrier Opening Increased Evan's Blue and IGF1 in the Spinal Cord of Healthy Mice

BSBC disruption was confirmed using Evan's Blue dye (EBD) and led to enhanced concentrations of IGF1, as shown in FIG. 2b in the spinal cord of healthy mice. EBD concentrations were 3.4 times higher in sonicated segments compared to non-sonicated segments (142 ug/g±24 vs 41 ug/g±8, p<0.001). IGF1 concentrations were 1100 times higher in sonicated regions (176±32 vs 0.16±0.008, p<0.0001), confirming that BSCB disruption leads to higher drug concentrations in the spinal cord.

### US Prolonged Survival of ALS SOD1 Mice

IGF1 was previously shown to be a potential neurotrophic factor and demonstrated beneficial effects when injected with retrograde viral delivery to reach motor neurons. Since IGF1 does not normally cross the BBB/BSCB, it was chosen to test whether US enhanced delivery of IGF1 increases survival in an ALS SOD1 mouse model.

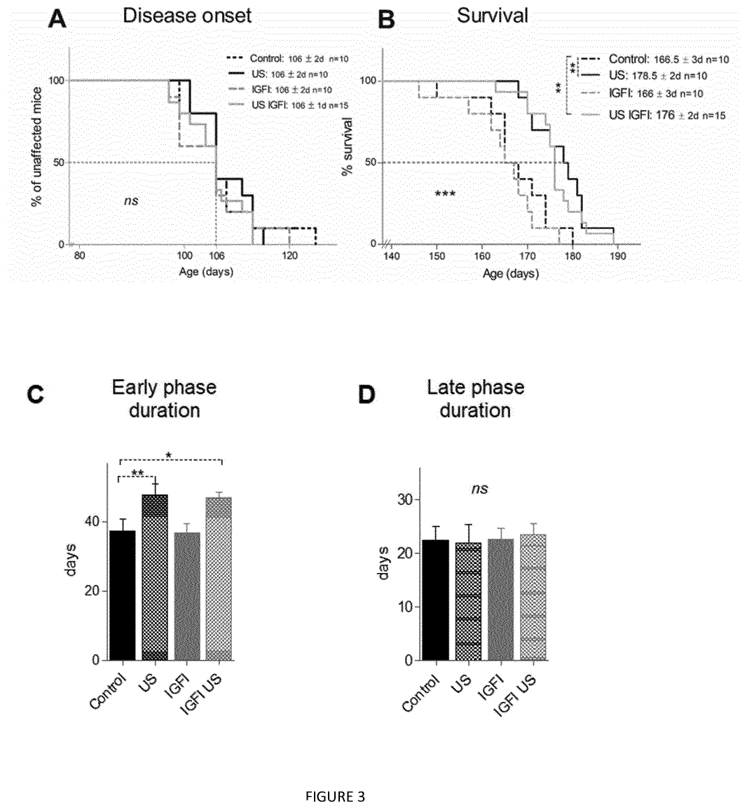

ALS mouse survival was increased in the group treated with IGF1 and US (US IGF1) compared to the IGF alone treatment (176 vs 166 days, p=0.019) or to the control group that did not receive IGF1 or US (176 vs 166.5 days, p=0.017, FIG. 3B). However, the US only group (that did not receive IGF1) also showed a similar survival improvement compared to the control group (178.5 vs 166.5 days, p=0.018) showing that the US treatment led to the longest survival (FIG. 3B).

There was no difference in age of mice at disease onset (FIG. 3A, median=106 days, p=0.82) and the increase of survival in the SOD1 model with either US or US+IGF1 was due to an effect on the early disease phase duration, which was significantly prolonged in sonicated groups compared to the control group (US=47.8±3.2, US IGF1=47±1.6, Control=37.4±3.4, p<0.05, FIG. 3C). In the IGF1 alone group, the duration of early phase was unaffected (36.8±2.8 vs 37.4±3.4). There was no difference between the different groups for the duration of the late phase of the disease (FIG. 3D). The last treatment was 28 days after the theoretical disease onset (P135) and all treatments occurred during the early phase of the disease.

### Repeated Blood Spinal Cord Barrier Opening by Ultrasound was Well Tolerated

Wild-type mice showed no signs of immediate or cumulative toxicity after five sonications. Body weight and motor function were comparable between sonicated and non-sonicated mice. In the 20 WT mice with repeated procedures (total of 94 procedures), two mice (1 sonicated and 1 non-sonicated) died of retro-orbital injection complications.

Four of the 50 ALS mice (US or US+IFG1) that received a total of 242 BSCB opening procedures had complications potentially related to the sonication (1.7% of procedures). One mouse (US) presented a paraplegia after the third treatment. One mouse (US+IGF1) presented a partially regressive motor deficit of one lower limb after the third treatment. Two mice (US and US+IGF1) presented totally regressive motor deficit of one lower limb, also after the third treatment. In addition, 7 ultrasound not related death occurred due to repeated intravenous injections and general anesthesia (2.9% of procedures). Deaths were distributed between groups and were not linked to any one intervention

Disease Progression Did not Correlate with SOD1 Burden

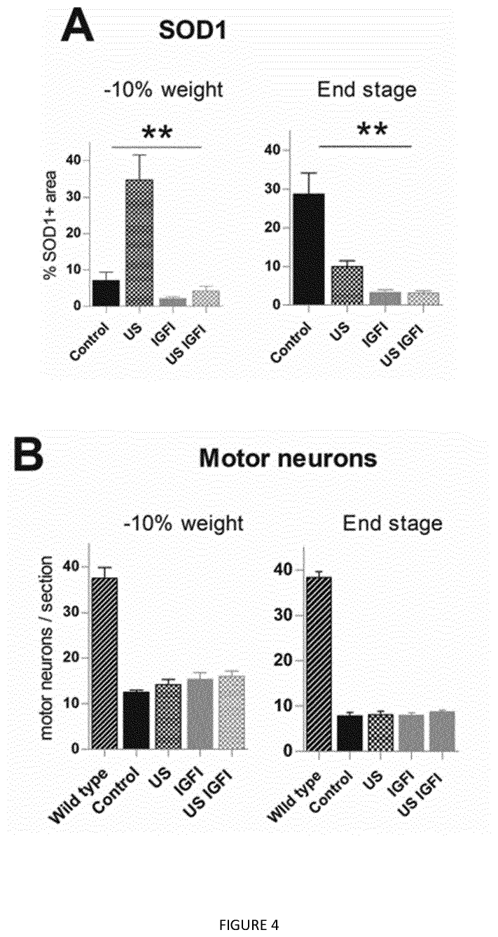

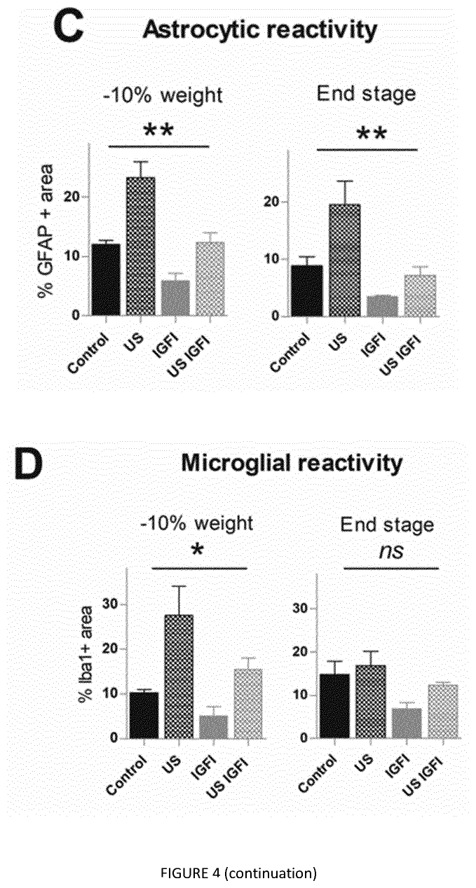

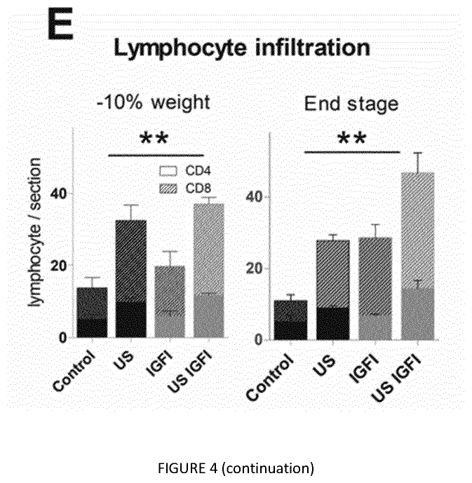

The immunofluorescent area occupied by SOD1 was higher in end stage SOD1^{G93A} mice compared to symptomatic mice (as expected, FIG. 4A. However, in symptomatic mice, the highest content of SOD1 (with a high spread) was measured in the US group (FIG. 4A). In addition, both in symptomatic and end stage mice, the IGF1 only group showed the lowest percentage of SOD1, comparable to US IGF1 group (FIG. 4A). Thus, there was no correlation between survival and SOD1 burden between treatment groups observed.

### US Modified Glial Cell Reactivity and Lymphocyte Infiltration

There was no significant difference between the different groups of ALS mice for motor neuron counts at the symptomatic stage (p=0.08) or at end stage (p=0.57), (FIG. 4B) in agreement with mice being at the same disease stage, as previously shown.

At both the symptomatic and end stage, there was a statistically significant difference of immunoreactive area for GFAP (astrocyte staining) between the different groups (symptomatic: p=0.007, end stage: p=0.005). Sonicated mice had a higher astrocytic activation compared to non-sonicated mice. (US: 23.2±2.8, Control: 12±2.6, US-IGFI: 12.4±8, IGF1: 5.9±2.5) (FIG. 4C). US treatment alone led to the highest increase in astrocyte reactivity (FIG. 4C) while IGF1 treatment alone led to a decrease compared to controls.

Microglial reactivity, measured by Iba1 immunoreactivity, was increased between the different groups (p=0.02) only at the symptomatic stage, corresponding approximatively to the end of the intervention (FIG. 4D, FIG. 1). This increase was the highest in sonicated only mice compared to the control group (US: 27.5±6.5% vs control: 10.3±0.6%) and was lower with IGF1 treatment (US IGF1: 15.5±2.6% and IGFI: 5±2.1%, FIG. 4D). At disease end stage, more than a month after the end of the intervention, no difference was measured in microglial reactivity in the US group (FIG. 4A).

Since the intervention transiently opened the BSCB, glial cells were reactive and as CD4+ T cells are known to be protective in this ALS mouse model the extent of lymphocyte infiltration between the different groups was further examined. At the symptomatic stage, there was a difference in lymphocyte (CD3+) infiltration number (p=0.01) with a higher number of lymphocytes per section in sonicated mice (US 34±3.8, US IGF1 37±2.2) compared to non-sonicated mice (Control: 13±3.5, IGF1: 20±5.3). This difference persisted at the end stage as well (p=0.003). (US: 28±2.1, US IGF1: 48±7.3, Control: 11±3.6, IGF1: 28.5±3.8) (FIG. 4B). In addition, CD4+ T cell numbers were quantified in sections. At the symptomatic stage, the number of CD4+ T cells was higher in the US alone and US+IGF1 groups compared to controls (US alone: 9.7±1.2 vs 5.1±1.2, p=0.045; US+UGF1: 11.7±0.6 vs 5.1±1.2, p=0.0018).

### CONCLUSIONS

These results demonstrate a beneficial effect of ultrasound-mediated blood-spinal cord barrier (BSCB) disruption on the progression of ALS in a mouse model. Treatments were started at the beginning of disease onset and not at a presymptomatic stage to mimic a potential therapeutic intervention as it would be performed in humans. Mice treated with five weekly ultrasound-based treatments to disrupt the BSCB led to the longest survival increase of any of the treatment groups (+12 days) and demonstrates that this type of intervention, without any co-administered drug therapies, may be a promising therapeutic strategy for this disease. The 12 day increase in survival was mainly due to the prolongation of the early symptomatic phase of the disease (+10 days), during which the treatment was administrated. A 12-day increase in survival in this model is similar to preclinical results reported with presymptomatic administration of riluzole, the current standard of care for this disease. This suggests an immediate effect of the ultrasound in delaying disease progression.

Motor functions after disease onset were preserved in the US-treated groups even though the usual decrease of motor function of SOD1^{G93A} mice is very rapid. Mice treated with ultrasound reached the end stage at an older age, 12 days after non-sonicated mice, and this stage is defined by an inability for the mouse to straighten itself. This indicates that US can delay clinical disease progression during the symptomatic phase in SOD1^{G93A} mice. Motor neuron loss was slower in the US and US-IGF1 groups compared with controls, since similar motor neuron counts were obtained at older age (+10 days) in sonicated mice.

SOD1^{G93A} mice, which were used as the ALS model in this study, are a particularly severe model as they rapidly present ALS symptoms. These mice quickly become too weak to tolerate general anesthesia, which led us to stop treatments at 135 days of age, and thus allowed for only a short window for therapeutic intervention of five weeks (5 sessions). Therefore, the potential benefit of the treatment if performed or continued into the late phase of the disease could not be assessed as these mice were unable to tolerate continued treatments.

There was no additional effect of IGF1 administration (whether or not associated with ultrasound) on survival or functional tests. The lack of efficacy of IGF1 may be explained by the administration scheme. Injections of IGF1 occurred only once a week, for a total of five injections. This administration scheme differs significantly from the regimen usually used clinically in humans (twice a day) or that previously found to be effective in mouse models. In addition, the positive IGF1 results obtained in a study in ALS mice were in a study in which IGF1 was uptaken by motor neurons via a viral vector, leading to continuous exposure to IGF1. The dose chosen (0.5 mg/kg) was an intermediate dose between that used in the clinic (human clinical trial: 0.05 mg/kg, twice per day) and that described as deleterious in this mouse model with subcutaneous infusion (3 mg/kg).

IGF1 measurements show that ultrasound treatment significantly increased the local IGF1 concentration by a thousand-fold while only a three-fold increase was observed for EBD. This difference can be explained by the fact that IGF1 is smaller in molecular weight (7.6 kDa) compared to EBD which binds with albumin forming a 68 kDa complex. It can be assumed that the intramedullary concentration of IGF1 may have been too high. Another hypothesis is that the intravascular concentration of endogenous IGF1 (or other factors) would be sufficient to exert a neuroprotective effect when the BSCB is opened. Thus, the contribution of additional IGF1 does not have any benefit.

The protective effect of lymphocytes in ALS mice is well known, as crossing SOD1^{G93A} mice with Rag2−/− mice (that do not produce any lymphocyte) accelerates disease progression. This protective effect was associated with CD4+ cells while involvement of CD8+ cells on ALS mouse survival was either not associated (in SOD1^{G93A} mice crossed with CD8−/− mice) or showed no effect (using anti-CD8+ blocking antibodies). In addition, a lower presence of CD4+ T cells were correlated with fast progression in ALS patients. It was observed that ultrasound treatment causes lymphocyte infiltration with an overall increase in the number of lymphocytes including CD4 lymphocytes. The migration of antigen-specific CD4+ T lymphocytes from the periphery to the CNS can also modify the inflammatory responses of resident glial cells as well as neuronal survival.

We also observed an increased activation of glial cells (microglial and astrocytic). This temporary effect has already been observed in pre-clinical studies investigating ultrasound-based BBB opening. Localized BBB opening in non-human primates (NHPs) showed that LIPU treatment triggers a short-lived immune response within the targeted region with increased microglia density around blood vessels detected on day 2 and resolved by day 18.

In addition, it is known that the entry of cytokines into the CNS causes recruitment of peripheral macrophages, which are difficult to distinguish from microglial activation. Microglial activation and macrophages play a complex role in the progression of the disease, and can be both protective and neurotoxic. Here, it was shown that the cumulative results of these changes led to an improvement in survival.

Endogenous neurotrophic factors such as GDNF and BDNF are known to have a neuroprotective effect in ALS. However, these peptides cannot cross the BSCB. Ultrasound-based opening of the BSCB could allow these physiologically circulating molecules in the blood to pass through and thus provide neuroprotection. LIPU has been shown to induce neuroprotective effects against cognitive dysfunction and brain injury in rats by increasing BDNF protein levels in the brain. It also attenuated the proinflammatory responses in microglia induced by LPS. A beneficial effect of ultrasound alone has already been observed in Alzheimer's disease both behaviorally and histologically reduction in amyloid plaques and tau protein.

After the procedure at the symptomatic stage, we observed an increase in mutant SOD1 protein in the CNS of sonicated mice. Since human SOD1 is ubiquitously expressed in the SOD1^{G93A} mouse, we could not assess whether the protein was endogenously more expressed by cells in the CNS or if it originated from the periphery and was released in the CNS after BSCB opening. At the end stage, while mutant SOD1 protein increased in the spinal cord compared to at the symptomatic stage, we observed a lower quantity of mutant SOD1 protein in the CNS of sonicated mice, suggesting a clearance phenomenon. The clearance of intracellular inclusions after sonications has already been observed for the tau protein in Alzheimer's disease models. The mechanism for intracellular protein clearance remains elusive but it is interesting to note that it occurs concomitantly with immune activation. Since Mutated SOD1 causes neurodegeneration through a toxic gain of function, it is possible that the reduction of SOD1 quantity we observed after this transient increase partially explains the prolonged survival of sonicated mice.

Therefore, these results confirm that the neurovascular barrier can be safely and repeatedly opened with low intensity pulsed ultrasound in an ALS mouse model. Furthermore, repeated transient BSCB disruption of ALS mouse model leads to an increase in survival and slowing down of motor neuron loss. These results demonstrate the potential of this technology alone or in combination with novel drug therapies for the treatment of ALS.

1. Ultrasound contrast agent for use in treating Amyotrophic Lateral Sclerosis (ALS) of a subject in need thereof, by transiently disrupting a neurovascular barrier of the subject, wherein the ultrasound contrast agent is administered before and/or during the application to the central nervous system of the subject, of at least one ultrasound (US) beam, wherein at least five sessions of application of the at least one US beam are performed repetitively.

2. Ultrasound contrast agent for use in treating ALS according to claim 1, wherein at least 5 cm³ of the neurovascular barrier is disrupted.

3. Ultrasound contrast agent for use in treating ALS according to claim 1, wherein a lag period between two successive sessions is of at least one week, preferably at least two weeks, and preferably of at most 9 weeks.

4. Ultrasound contrast agent for use in treating ALS according to claim 1, wherein the ultrasound (US) beam is applied to the spinal cord of the subject, in order to transiently disrupt the blood-spinal cord barrier (BSCB) of the subject.

5. Ultrasound contrast agent for use in treating ALS according to claim 4, wherein the at least one US beam targets a cervical portion of the spinal cord of the subject, preferably in a portion between neurological metameric segments C3 and C7, and/or a lumbar portion of the spinal cord of the subject, preferably in a portion between neurological metameric segments L1 and S4.

6. Ultrasound contrast agent for use in treating ALS according to claim 4, wherein the at least one US beam is applied by use of an external US transducer or a US transducer previously implanted inside the spinal canal of the spine of the subject, preferentially in the subdural and/or in the epidural space of the spinal canal.

7. Ultrasound contrast agent for use in treating ALS according to claim 1, wherein the US beam is applied to the brain of the subject, in order to transiently disrupt the blood brain barrier (BBB) of the subject.

8. Ultrasound contrast agent for use in treating ALS according to claim 7, wherein the at least one US beam targets a region within the right and/or left frontal lobes, more particularly a region within the primary motor cortex area and/or the premotor cortex such as supplementary motor cortex area.

9. Ultrasound contrast agent for use in treating ALS according to claim 7, wherein the at least one US beam is applied by use of a US transducer that has been previously implanted within a skull thickness of the subject.

10. Ultrasound contrast agent for use in treating ALS according to claim 1, wherein the at least one US beam is focused or unfocused.

11. Ultrasound contrast agent for use in treating ALS according to claim 1, wherein the ultrasound contrast agent is administered in absence of a therapeutically active agent.

12. Ultrasound contrast agent for use in treating ALS according to claim 1, wherein no therapeutically active agent is administered to the subject before, during or after disruption of the neurovascular barrier.

13. Ultrasound contrast agent for use in treating ALS according to claim 1, wherein the ultrasound contrast agent is administered in conjunction with a therapeutically active agent.

14. Ultrasound contrast agent for use in treating ALS according to claim 1, wherein therapeutically active agent is administered to the subject either before, after or during disruption of the neurovascular barrier (local action), or either between two disruption sessions (systemic action).

15. Ultrasound contrast agent for use in treating ALS according to claim 1, wherein several US beams are applied sequentially or in synchrony, for a period of time between 100 and 500 seconds, or for a period of time of 250 seconds.

16. Ultrasound contrast agent for use in treating ALS according to claim 1, wherein the at least one US beam is applied with a resonance frequency ranging from 1 to 1.1 MHz, preferably at 1.05 MHz.

17. Ultrasound contrast agent for use in treating ALS according to claim 1, wherein the at least one US beam is applied in pulses of duration ranging from 10 to 50 ms, from 20 to 30 ms, or at 25 ms, and with a pulse repetition frequency ranging from 0.3 to 1.2 Hz, or at 1 Hz.

18. Ultrasound contrast agent for use in treating ALS according to claim 1, wherein the at least one US beam is applied with a pressure level ranging from 0.3 to 1.2 MPa, preferably or at 1 MPa.

19. Ultrasound contrast agent for use in treating ALS according to claim 1, wherein the dose of ultrasound contrast agent administered to the subject is approximately 0.05 to 0.2 ml/kg.


SEQUENCE LISTING (3 sequences)

SEQ ID NO: 1 - DNA, 30 bp, ARTIFICIAL SEQUENCE
  SOD1 forward primer
gttacatataggggtttacttcataatctg

SEQ ID NO: 2 - DNA, 30 bp, ARTIFICIAL SEQUENCE
  SOD1 forward primer
ccaagatgcttaactcttgtaatcaatggc

SEQ ID NO: 3 - DNA, 31 bp, ARTIFICIAL SEQUENCE
  mouse and human SOD1 reverse primer
cagcagtcacattgcccaggtctccaacatg